(19) United States
(12) Patent Application Publication
(10) Pub. No.: US 2020/0197642 A1
(43) Pub. Date: Jun. 25, 2020

(54) PRESSURE SUPPORT SYSTEM AND METHOD OF PROVIDING PRESSURE SUPPORT THERAPY TO A PATIENT

(71) Applicant: KONINKLIJKE PHILIPS N.V., Eindhoven (NL)

(72) Inventors: MICHAEL THOMAS KANE, HARRISON CITY, PA (US); SIDNEY CANNON-BAILEY, MONROEVILLE, PA (US); MARK LITTLEFIELD, WEXFORD, PA (US)

(21) Appl. No.: 16/724,587

(22) Filed: Dec. 23, 2019

Related U.S. Application Data
(60) Provisional application No. 62/784,588, filed on Dec. 24, 2018.

(51) Int. Cl. A61M 16/00; A61B 5/087

(52) U.S. Cl. CPC A61M 16/026; A61M 16/0003; A61B 5/087

(57) ABSTRACT

A pressure support system (2) for providing pressure support therapy to a patient. The pressure support system includes an airflow generator (6) structured to generate a flow of breathing gas to the patient, a number of sensors (22,27) structured to sense characteristics of breaths of the patient, and a processing unit (24) structured to calculate a number of breath features of the patient based on the characteristics of breaths of the patient, to calculate a comfort level based on one or more of the calculated number of breath features, and to adjust a gain of the airflow generator based on the calculated comfort level.

### CROSS-REFERENCE TO PRIOR APPLICATIONS

This application claims the benefit of U.S. application Ser. No. 62/784,588, filed Dec. 24, 2018. This of which are incorporated herein by references.

### BACKGROUND OF THE INVENTION

### 1. Field of the Invention

The present invention pertains to a pressure support system, and, in particular, to a pressure support system that adjusts provided pressure based on breath features of a patient.

### 2. Description of the Related Art

Many individuals suffer from disordered breathing during sleep. Sleep apnea is a common example of such sleep disordered breathing suffered by millions of people throughout the world. One type of sleep apnea is obstructive sleep apnea (OSA), which is a condition in which sleep is repeatedly interrupted by an inability to breathe due to an obstruction of the airway; typically the upper airway or pharyngeal area. Obstruction of the airway is generally believed to be due, at least in part, to a general relaxation of the muscles which stabilize the upper airway segment, thereby allowing the tissues to collapse the airway. Another type of sleep apnea syndrome is a central apnea, which is a cessation of respiration due to the absence of respiratory signals from the brain's respiratory center. An apnea condition, whether OSA, central, or mixed, which is a combination of OSA and central, is defined as the complete or near cessation of breathing, for example a 90% or greater reduction in peak respiratory air-flow.

Those afflicted with sleep apnea experience sleep fragmentation and complete or nearly complete cessation of ventilation intermittently during sleep with potentially severe degrees of oxyhemoglobin desaturation. These symptoms may be translated clinically into extreme daytime sleepiness, cardiac arrhythmias, pulmonary-artery hypertension, congestive heart failure and/or cognitive dysfunction. Other consequences of sleep apnea include right ventricular dysfunction, carbon dioxide retention during wakefulness, as well as during sleep, and continuous reduced arterial oxygen tension. Sleep apnea sufferers may be at risk for excessive mortality from these factors as well as by an elevated risk for accidents while driving and/or operating potentially dangerous equipment.

Even if a patient does not suffer from a complete or nearly complete obstruction of the airway, it is also known that adverse effects, such as arousals from sleep, can occur where there is only a partial obstruction of the airway. Partial obstruction of the airway typically results in shallow breathing referred to as a hypopnea. A hypopnea is typically defined as a 50% or greater reduction in the peak respiratory air-flow followed by oxyhemoglobin desaturation and/or a cortical arousal. Other types of sleep disordered breathing include, without limitation, upper airway resistance syndrome (UARS) and vibration of the airway, such as vibration of the pharyngeal wall, commonly referred to as snoring.

It is well known to treat sleep disordered breathing by applying a positive airway pressure (PAP) to the patient's airway using an airway pressure support system that typically includes a mask, a pressure generating device, and a conduit to deliver positive pressure breathing gas from the pressure generating device to the patient through the mask. This positive pressure effectively “splints” the airway, thereby maintaining an open passage to the lungs. In one type of PAP therapy, known as continuous positive airway pressure (CPAP), the pressure of gas delivered to the patient is constant throughout the patient's breathing cycle. It is also known to provide a positive pressure therapy in which the pressure of gas delivered to the patient varies with the patient's breathing cycle, or varies with the patient's effort, to increase the comfort to the patient. This pressure support technique is referred to as bi-level pressure support, in which the inspiratory positive airway pressure (IPAP) delivered to the patient is higher than the expiratory positive airway pressure (EPAP). It is further known to provide a positive pressure therapy in which the pressure is automatically adjusted based on the detected conditions of the patient, such as whether the patient is experiencing an apnea and/or hypopnea. This pressure support technique is referred to as an auto-titration type of pressure support, because the pressure support device seeks to provide a pressure to the patient that is only as high as necessary to treat the disordered breathing.

Pressure support therapies as just described involve the placement of a patient interface device including a mask component having a soft, flexible sealing cushion on the face of the patient. The mask component may be, without limitation, a nasal mask that covers the patient's nose, a nasal/oral mask that covers the patient's nose and mouth, or a full face mask that covers the patient's face. Such patient interface devices may also employ other patient contacting components, such as forehead supports, cheek pads and chin pads. The patient interface device is typically secured to the patient's head by a headgear component. The patient interface device is connected to a gas delivery tube or conduit and interfaces the pressure support device with the airway of the patient, so that a flow of breathing gas can be delivered from the pressure/flow generating device to the airway of the patient.

It is important that pressure support therapy is comfortable for a patient. Uncomfortable pressure support therapy can dissuade a patient from continuing with the therapy. For example, a patient may be very compliant with PAP therapy, but has the feeling that when he wakes up he does not feel good or does not feel refreshed. This patient may suspect that the therapy is either not working, or somehow negatively influencing their sleep. Another example is a non-compliant patient. Non-compliance may be due to many reasons, one of which arising from the disbelief by the patient that the therapy works for him/her, despite the information from the referring physician. Each time he uses the PAP therapy, he has the feeling that he sleeps worse than when not using it and therefore refuses to continue with a regular therapy.

A patient's comfort can be affected by the level of pressure compensation provided to the patient. Components in the pressure support system such as components in a patient circuit between the pressure generating device and the patient interface device, as well as the patient interface device itself, can affect the level of pressure provided to the patient. Additionally, characteristics of the patient can affect the pressure level felt by the patient. A pressure support system can initially be setup to compensate for the components of the patient circuit and patient interface device, but changes in the components of the system or changes in the patient can lead to a pressure support therapy regimen that, while previously comfortable to the patient, is no longer comfortable. Too much pressure compensation can cause the patient to feel as if the device is forcing him/her to breathe during inhalation and can cause the patient to feel as if pressure is dropping away during exhalation. Too little pressure can cause the patient to feel as if it is hard to breathe during inhalation and can cause the patient to feel as if it is hard to exhale during exhalation.

### SUMMARY OF THE INVENTION

A pressure support system for providing pressure support therapy to a patient, the pressure support system comprises: an airflow generator structured to generate a flow of breathing gas to the patient; a number of sensors structured to sense characteristics of breaths of the patient; and a processing unit structured to calculate a number of breath features of the patient based on the characteristics of breaths of the patient, to calculate a comfort level based on one or more of the calculated number of breath features, and to adjust a gain of the airflow generator based on the calculated comfort level.

A method of providing pressure support therapy to a patient comprises: generating a flow of breathing gas to the patient; sensing characteristics of breaths of the patient; calculating a number of breath features of the patient based on the characteristics of breaths of the patient; calculating a comfort level based on one or more of the calculated number of breath features; and adjusting a gain of the flow of breathing gas to the patient based on the calculated comfort level.

A non-transitory computer readable medium storing one or more programs, including instructions, which when executed by a computer, causes the computer to perform a method of providing pressure support therapy to a patient. The method comprises: generating a flow of breathing gas to the patient; sensing characteristics of breaths of the patient; calculating a number of breath features of the patient based on the characteristics of breaths of the patient; calculating a comfort level based on one or more of the calculated number of breath features; and adjusting a gain of the flow of breathing gas to the patient based on the calculated comfort level.

### BRIEF DESCRIPTION OF THE DRAWINGS

FIG. 1 is a schematic diagram of an airway pressure support system according to an exemplary embodiment of the disclosed concept;

FIG. 2 is a schematic diagram of a portion of a pressure support system according to an exemplary embodiment of the disclosed concept;

FIG. 3 is a flowchart of a method of providing pressure support therapy to a patient in accordance with an exemplary embodiment of the disclosed concept;

FIG. 4 is a flowchart of a method of providing pressure support therapy to a patient in accordance with another exemplary embodiment of the disclosed concept; and

FIG. 5 is a graph showing the association of a perceived comfort level and gain in accordance with an exemplary embodiment of the disclosed concept.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

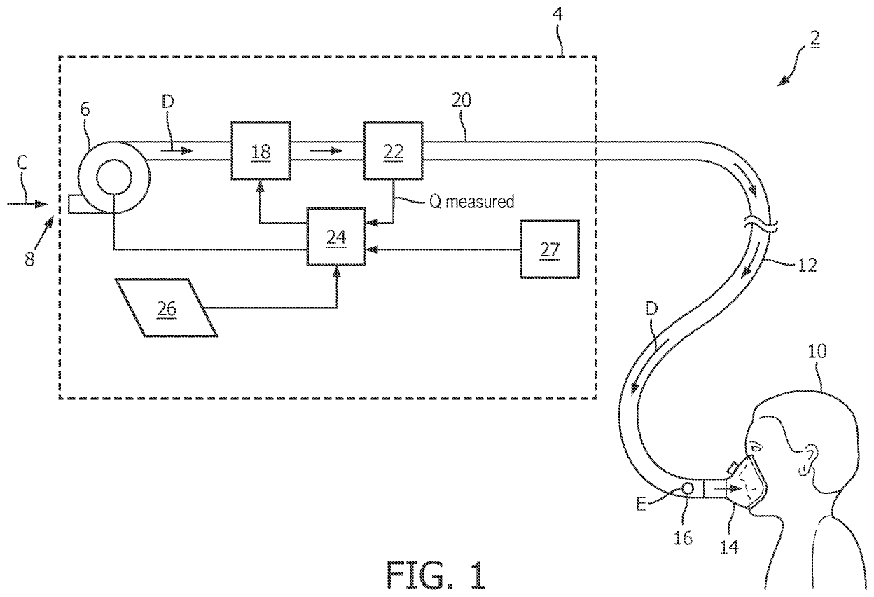

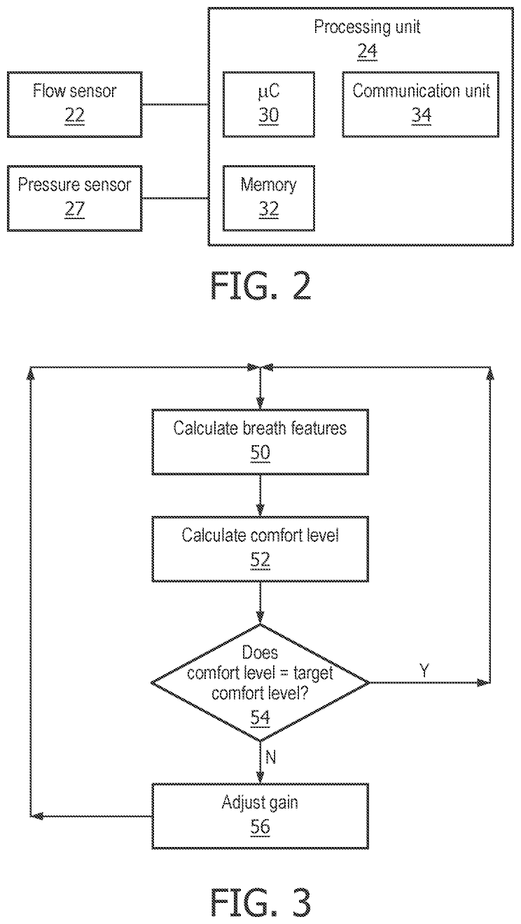

FIG. 1 is a schematic diagram of an airway pressure support system 2 according to one particular, non-limiting exemplary embodiment in which the present invention may be implemented. Referring to FIG. 2, airway pressure support system 2 includes a pressure support device 4 which houses an airflow generator 6, such as a blower used in a conventional CPAP or bi-level pressure support device. Pressure generator 6 receives breathing gas, generally indicated by arrow C, from the ambient atmosphere through a filtered air inlet 8 provided as part of pressure support device 4, and generates a flow of breathing gas therefrom for delivery to an airway of a patient 10 at relatively higher and lower pressures, i.e., generally equal to or above ambient atmospheric pressure, to generate pressure to provide pressure compensation to patient 10 via a patient circuit 12,14. In the exemplary embodiment, airflow generator 6 is capable of providing a flow of breathing gas ranging in pressure from 3-30 cmH2O. The pressurized flow of breathing gas from airflow generator 6, generally indicated by arrow D, is delivered via a delivery conduit 12 to a breathing mask or patient interface 14 of any known construction, which is typically worn by or otherwise attached to patient 10 to communicate the flow of breathing gas to the airway of patient 10. Delivery conduit 12 and patient interface device 14 are typically collectively referred to as the patient circuit.

Pressure support system 2 shown in FIG. 1 is what is known as a single-limb system, meaning that the patient circuit includes only delivery conduit 12 connecting patient 10 to pressure support system 2. As such, an exhaust vent 16 is provided in delivery conduit 12 for venting exhaled gases from the system as indicated by arrow E. It should be noted that exhaust vent 16 can be provided at other locations in addition to or instead of in delivery conduit 12, such as in patient interface device 14. It should also be understood that exhaust vent 16 can have a wide variety of configurations depending on the desired manner in which gas is to be vented from pressure support system 2.

The present concept also contemplates that pressure support system 2 can be a two-limb system, having a delivery conduit and an exhaust conduit connected to patient 10. In a two-limb system (also referred to as a dual-limb system), the exhaust conduit carries exhaust gas from patient 10 and includes an exhaust valve at the end distal from patient 10. The exhaust valve in such an embodiment is typically actively controlled to maintain a desired level or pressure in the system, which is commonly known as positive end expiratory pressure (PEEP).

Furthermore, in the illustrated exemplary embodiment shown in FIG. 1, patient interface 14 is a nasal/oral mask. It is to be understood, however, that patient interface 14 can include a nasal mask, nasal pillows, a tracheal tube, an endotracheal tube, or any other device that provides a suitable gas flow communicating function. Also, for purposes of the present invention, the phrase “patient interface” can include delivery conduit 12 and any other structures that couple the source of pressurized breathing gas to patient 10.

In the illustrated embodiment, pressure support system 2 includes a pressure controller in the form of a valve 18 provided in internal delivery conduit 20 provided in a housing of pressure support device 4. Valve 18 controls the pressure of the flow of breathing gas from airflow generator 6 that is delivered to patient 10. For present purposes, airflow generator 6 and valve 18 are collectively referred to as a pressure generating system because they act in concert to generate and control the pressure and/or flow of gas delivered to patient 10. However, it should be apparent that other techniques for controlling the pressure of the gas delivered to patient 10, such as varying the blower speed of airflow generator 6, either alone or in combination with a pressure control valve, are contemplated by the present invention. Thus, valve 18 is optional depending on the technique used to control the pressure of the flow of breathing gas delivered to patient 10. If valve 18 is eliminated, the pressure generating system corresponds to airflow generator 6 alone, and the pressure of gas in the patient circuit is controlled, for example, by controlling the motor speed of airflow generator 6.

Pressure support system 2 further includes a flow sensor 22 that measures the flow of the breathing gas within delivery conduit 20 and delivery conduit 12. In the particular embodiment shown in FIG. 1, flow sensor 22 is interposed in line with delivery conduits 20 and 12, most preferably downstream of valve 18. Pressure support system 2 additionally includes a pressure sensor 27 that detects the pressure of the pressurized fluid in delivery conduit 20. While the point at which the flow is measured by flow sensor 22 and the pressure is measured by pressure sensor 27 are illustrated as being within pressure support device 4, it is to be understood that the location at which the actual flow and pressure measurements are taken may be anywhere along delivery conduits 20 or 12. The flow of breathing gas measured by flow sensor 22 and the pressure detected by pressure sensor 27 are provided to processing unit 24 to determine the flow of gas at patient 10 (Q_{PATIENT}).

Techniques for calculating Q_{PATIENT} are well known, and take into consideration the pressure drop of the patient circuit, known leaks from the system, i.e., the intentional exhausting of gas from the circuit as indicated by arrow E in FIG. 1, and unknown leaks from the system, such as leaks at the mask/patient interface. The present invention contemplates using any known or hereafter developed technique for calculating leak flow, and using this determination in calculating Q_{PATIENT} using measured flow and pressure. Examples of such techniques are taught by U.S. Pat. Nos. 5,148,802; 5,313,937; 5,433,193; 5,632,269; 5,803,065; 6,029,664; 6,539,940; 6,626,175; 6,920,875; and 7,011,091, the contents of each of which are incorporated by reference into the present invention.

Of course, other techniques for measuring the respiratory flow of patient 10 are contemplated by the present invention, such as, without limitation, measuring the flow directly at patient 10 or at other locations along delivery conduit 12, measuring patient flow based on the operation of gas flow generator 6, and measuring patient flow using a flow sensor upstream of valve 18.

An input/output device 26 is provided for setting various parameters used by pressure support system 2, as well as for displaying and outputting information and data to a user, such as a clinician or caregiver.

Processing unit 24 is structured to control airflow generator 6 to implement a pressure support therapy regimen for patient 10. Processing unit 24 is also structured to calculate breath features of patient 10 and control airflow generator 6 based on the calculated breath features. In an example embodiment, processing unit 24 is structured to calculate patient comfort based on a number of breath features and to control airflow generator 6 to optimize patient comfort. For example, patient comfort can be calculated on a scale ranging from starvation (too little pressure compensation), to optimal comfort, to over-ventilation (too much pressure compensation). If processing unit 24 calculates that patient comfort is in a starvation region based on the breath features, processing unit 24 may control airflow generator 6 to increase the gain (i.e. the pressure compensation) of the pressure support therapy provided to patient 10. Similarly, if processing unit 24 calculates that patient comfort is in an over-ventilation region based on the breath features, processing unit 24 may control airflow generator 6 to decrease the gain of the pressure support therapy provided to patient 10.

FIG. 2 is a schematic diagram of a portion of pressure support system 2 in accordance with an exemplary embodiment of the disclosed concept. Processing unit 24 in accordance with an exemplary embodiment of the disclosed concept is shown in more detail in FIG. 2.

Processing unit 24 includes a processor 30, a memory 32, and a communication unit 34. Processor 30 may form all or part of a processing portion which may be, for example, a microprocessor, a microcontroller or some other suitable processing device. Memory 32 may form all or part of a memory portion that may be internal to the processing portion or operatively coupled to the processing portion and provide a storage medium for data and software executable by the processing portion for implementing functionality of processing unit 23 and controlling the operation of pressure support system 2. Memory 32 can be any of one or more of a variety of types of internal and/or external storage media such as, without limitation, RAM, ROM, EPROM(s), EEPROM(s), FLASH, and the like that provide a storage register, i.e., a machine readable medium, for data storage such as in the fashion of an internal storage area of a computer, and can be volatile memory or nonvolatile memory.

Communication unit 34 may provide for communication between processing unit 24 and other components of pressure support device 4, components of the patient circuit, or other external devices. Communication unit 34 may also facilitate communication with external devices. For example and without limitation, communication unit 34 may facilitate communication with electronic devices such as a phone, tablet, computer, or other devices directly or via a network. Communication facilitated by communication unit 34 may allow processing unit 24 to send and/or receive data from the component or device it communicates with.

FIG. 3 is a flowchart of a method of controlling a pressure support system to optimize comfort in accordance with an example embodiment of the disclosed concept. The method may be implemented in pressure support system 2 of FIG. 1 or any other suitable pressure support system. At 50, breath features are calculated. The breath features may be calculated by, for example and without limitation, processing unit 24. At 52, a comfort level of a patient is calculated based on the breath features. At 54, it is determined whether the calculated comfort level is equal to a target comfort level. If the calculated comfort level is not equal to the target comfort level, the gain is adjusted at 56. After 56, the method returns to 50. For example and without limitation, processing unit 24 may control airflow generator 6 to increase or decrease the gain. If the calculated comfort level is equal to the target comfort level, the method returns to 50. By repeating the method, the comfort level is continuously calculated and the gain is adjusted to bring the comfort level to the target comfort level.

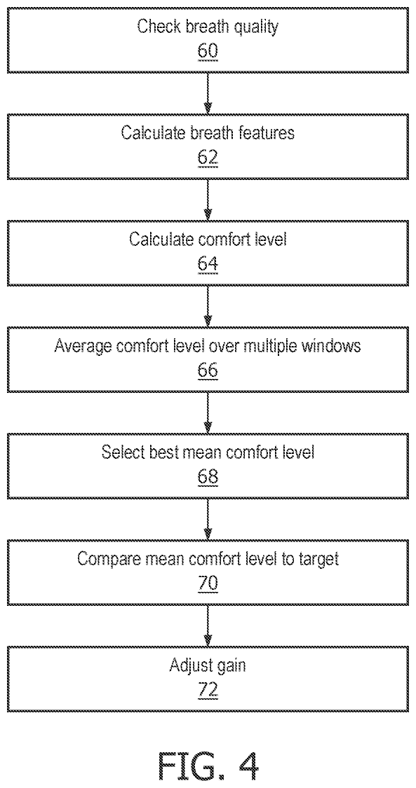

FIG. 4 is a flowchart of a method of controlling a pressure support system to optimize comfort in accordance with another example embodiment of the disclosed concept. The method may be implemented in pressure support system 2 of FIG. 1 or any other suitable pressure support system. At 60, breath quality is checked. The breath quality of the patient is evaluated to determine if it is clinically normal. In an example embodiment, breaths that do not meet a threshold quality are not processed for determining breath features. The quality of a breath may be based on the breath passing a number of test evaluations. Test evaluations may include, without limitation, a check that inspiration volume and expiration volume are within 50% of each other, a check that inspiration time and expiration time are within 70% of predetermined normal values, a check that maximum patient flow during inspiration is within 70% of minimum patient flow during expiration, a check that maximum patient flow during inspiration is greater than 10 lpm, a check that maximum patient flow during inspiration is less than 75 lpm, a check that inspiration volume is greater than 150 ml, a check that inspiration volume is less than 1800 ml, a check that expiration volume is greater than 150 ml, a check that expiration volume is less than 1800 ml, a check that inspiration time is greater than 0.5 s, a check that inspiration time is less than 2 s, a check that expiration time is greater than 0.5 s, and a check that expiration time is less than 2 s. A breath may be determined to meet the threshold quality if it passes a specified number of the test evaluations. The test evaluations are provided as an example of a set of test evaluations that may be employed. However, it will be appreciated that the example test evaluations may be modified without departing from the scope of the disclosed concept. It will also be appreciated that different test evaluations for determining breath quality may be employed without departing from the scope of the disclosed concept. Breaths that meet the threshold quality are further processed to determine breath features, while breaths that do not meet the threshold quality are omitted. In some example embodiments, 60 may be omitted.

At 62, breath features are calculated. In an example embodiment, the breath features are also normalized. Any suitable number and type of breath features may be calculated. In some embodiments, a select group of breath features that have been found to be correlated with a patient comfort level are calculated. At 64, a comfort level of the patient is calculated based on the calculated breath features. At 66, an average comfort level of the patient is calculated over multiple windows. For example, the average comfort level may be calculated over moving windows of 10, 20, and 30 breaths. However, it will be appreciated that any number or length of windows may be used without departing from the scope of the disclosed concept. It will also be appreciated that other statistical properties may be calculated such as, without limitation, median, range, standard deviation, etc.

At 68, the best mean comfort level is selected. In an example embodiment, the mean comfort level corresponding to the longest window from 66 is selected. For example, once the shortest window (e.g., 10 breaths) is filled, the medium window (e.g., 20 breaths) begins to fill and, once the medium window is filled, the long window (e.g., 30 breaths) begins to fill. The mean comfort level may be selected after a predetermined period of time and the mean comfort value that is selected may correspond to the longest window that filled during that predetermined period of time. In some embodiments, the predetermined period of time may be changed based on one or more conditions. For example, the statistical properties of the mean comfort level may indicate changes in system resistance (e.g., changes in components of the pressure support system such as a humidifier, tubing, or mask or changes in the patient such as nasal resistance or upper airway resistance). In response to sensing a change in system resistance, the predetermined period of time may be shortened in order to react quickly to the change. As an alternative to using a predetermined period of time, a particular window may be selected. For example, the shortest window may be selected in order to react quickly to a change. Once the shortest window is filled, the mean comfort value associated with the shortest window may be output.

At 70, the mean comfort level output at 66 is compared to a target comfort level. The target comfort level may come from one or many source such as, without limitation, a predetermined comfort level associated with the pressure support system or a user selected comfort level. In an example embodiment, the target comfort level may be generated based on experimental data. At 72, the gain of the airflow generator is adjusted to drive the mean comfort level toward the target comfort level. For example, if the mean comfort level is in the starvation region, the gain of the airflow generator may be increased to drive the mean comfort level toward a target comfort level in the comfortable region. It will be appreciated that the gain may be limited to gain levels between minimum and maximum levels associated with the pressure support system.

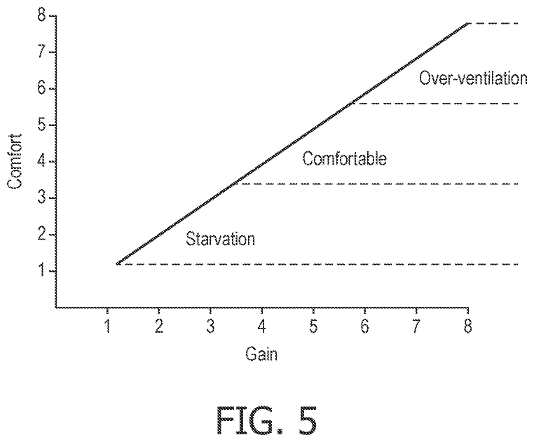

FIG. 5 is a graph showing an example of comfort level perceived by a patient as related to a gain (i.e., compensation) provided by an airflow generator. In the graph of FIG. 5, a perceived comfort level of 4 is ideal and in a comfortable range for the patient. Higher perceived comfort levels correspond to an over-ventilation regions and lower perceived comfort levels correspond to a starvation region. As shown in FIG. 5, as the gain is increased, the perceived comfort level moves from the starvation region to the comfortable region to the over-ventilation region. A gain of 4 corresponds to the ideal perceived comfort level of 4. In the graph of FIG. 5, the gain values are representative and the actual gain provided by an airflow generator will be proportional to the gain values shown in FIG. 5.

While the graph shown in FIG. 5 represents an ideal relation between perceived comfort and gain, it has practical limitations. Components of a pressure support system and conditions of the patient themselves introduce system resistance that must be compensated for. For example, when system resistance is introduced, the gain should be increased to compensate for the system resistance. When a pressure support system is initially configured, the system resistance can be determined and the gain can be set to compensate for the system resistance to provide a gain corresponding to an ideal perceived comfort level. However, if any components are changed, the system resistance is changed and the gain should be adjusted to compensate for the changed system resistance. In conventional pressure support systems, such adjustment or recalibration was done manually and requires knowing what components are in the pressure support system and what system resistance they introduce. For example, adding a bacteria filter would introduce a known system resistance that would then be compensated for the next time the system was calibrated. However, even with manual recalibration after changing system components, conventional pressure support systems cannot continuously compensate for system resistance caused by the patient themselves. For example, upper and lower airway resistance of a patient are components of system resistance. A patient becoming congested would increase system resistance. In conventional pressure support systems, this increase would not be compensated for and the perceived comfort of the patient would decrease.

In the present disclosed concept, an association between the perceived comfort level of the patient and breath features of the patient has been determined. In this manner, a number of breath features of the patient can be used to calculate the comfort level of the patient. The gain can then be adjusted to drive the comfort level of the patient toward a target comfort level. The breath features and their association with the perceived comfort level of the patient will be described hereinafter.

A number of breath features of a patient can be calculated based on outputs of sensors in a pressure support system, such as the outputs of flow and pressure sensors 22, 27 in the pressure support system 2 of FIG. 1. From the outputs of flow and pressure sensors 22,27, the flow to the patient can be determined. Once the flow to the patient has been determined, numerous breath features can be calculated. Some general breath features that are often used by clinicians to describe respiration of a patient are tidal volume of inspiration, tidal volume of expiration, peak flow amplitude of inspiration, peak flow amplitude of expiration, required time to deliver 0.707 of total volume for inspiration, and required time to deliver 0.707 to total volume for expiration. Numerous other breath features can be derived from the outputs of flow and pressure sensors 22,27.

In the present disclosed concept, an association between breath features and perceived comfort level has been determined. The relation is based on Equation 1:

Y=M1X1+M2X2+M3X3 . . . +B Equation 1

In Equation 1, Y is the perceived comfort level, X1, X2, X3, etc. are each values of breath features (which may be normalized in some example embodiments), M1, M2, M3, etc. are coefficients corresponding to the breath features, and B is a bias value. Equation 1 represents a multiple linear regression which describes a relationship of two more multiple input variables to one target output variable. The application of this technique is well known and suitable to this work. It is recognized that any number of additional techniques could be applied to model this relationship including advanced areas in artificial intelligence like neural networks.

In order to apply Equation 1, a study was performed on a number of patients. The patients provided their perceived comfort level during pressure support therapy and the breath features of the patients were monitored during the pressure support therapy. When this data is applied to Equation 1, Y and X1, X2, X3, etc. are known and M1, M2, M3, etc. and B are unknown. However, using data analysis, such as lasso regression or other machine learning techniques, values of M1, M2, M3, etc. and B can be derived from study data. Once M1, M2, M3 etc. and B have been determined, for a subsequent patient, their breath features during pressure support therapy can be calculated and their perceived comfort level can be calculated using Equation 1. For example, referring to FIG. 5, a patient's breath features may be monitored and, using Equation 1, their perceived comfort level is determined to be 2. According to FIG. 5, the comfort level of 2 is located in the starvation region. In response, the gain is increased to drive the patient's perceived comfort level toward the comfortable region.

It will be appreciated that any number of breath features can be used in Equation 1. However, in some embodiments of the disclosed concept, a select number of breath features are used. In one embodiment, the following breath features are used: asymmetry of the patient inspiration flow waveform, tidal volume of inspiration divided by time of inspiration, inspiration time divided by exhalation time, exhaled tidal volume divided by the minimum patient flow observed during exhalation, respiratory rate divided by tidal volume, duration in seconds required to inspire 67% of tidal volume of inspiration, and tidal volume of inspiration divided by maximum flow value observed during inspiration. In some embodiments, the following breath features are used: minimum patient flow during expiration divided by the pressure difference observed during expiration, maximum patient flow during inspiration divided by the pressure difference observed during inspiration, tidal volume of inspiration divided by the pressure difference during inspiration, and tidal volume of expiration divided by the pressure difference during expiration. It will be appreciated that the preceding lists of breath features, in whole, or in part, and Equation 1, may be employed in the pressure support system of FIGS. 1 and 2 or the methods of FIGS. 3 and 4 to calculate the comfort level of a patient.

The preceding lists of breath features use many breath features that are composites of multiple breath features. For example, many of the breath features are one breath feature divided by another breath feature. Using these types of composite breath features makes the breath features more robust against variations in characteristics of patients. It will be appreciated that the preceding lists of breath features are provided as an exemplary list of breath features that are associated with perceived patient comfort, but it will be appreciated that the disclosed concept is not limited to using such breath features. It will be appreciated that according to the disclosed concept, any set of breath features may be used to calculate the perceived comfort value of a patient.

In accordance with the disclosed concept, the patient's perceived comfort level can be calculated based on the patient's breath features and gain can be adjusted to drive the perceived comfort level to a target comfort level using the pressure support system 2 of FIGS. 1 and 2, the methods of FIGS. 3 and 4, or other suitable systems or methods. The perceived comfort level can be periodically calculated as the patient is receiving pressure support therapy. If components of the system are changed or the patient's condition causes a change in system resistance, the perceived comfort level based on the patient's breath features will change and the gain can be automatically adjusted to drive the perceived comfort level toward the target comfort level. Any changes in system resistance can be automatically compensated for rather than having a technician or other medical provider manually recalibrate the pressure support system.

It is contemplated that aspects of the disclosed concept can be embodied as computer readable codes on a tangible computer readable recording medium. The computer readable recording medium is any data storage device that can store data which can be thereafter read by a computer system. Examples of the computer readable recording medium include read-only memory (ROM), random-access memory (RAM), CD-ROMs, magnetic tapes, floppy disks, and optical data storage devices.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A pressure support system for providing pressure support therapy to a patient, the pressure support system comprising:
an airflow generator structured to generate a flow of breathing gas to the patient;
a number of sensors structured to sense characteristics of breaths of the patient; and
a processing unit structured to calculate a number of breath features of the patient based on the characteristics of breaths of the patient, to calculate a comfort level based on one or more of the calculated number of breath features, and to adjust a gain of the airflow generator based on the calculated comfort level.

2. The pressure support system of claim 1, wherein the comfort level of the patient is a representation of the patient's perceived comfort with a level of pressure compensation provided in the pressure support therapy.

3. The pressure support system of claim 1, wherein the number of breath features include at least one of: asymmetry of a patient inspiration flow waveform, tidal volume of inspiration divided by time of inspiration, inspiration time divided by exhalation time, exhaled tidal volume divided by a minimum patient flow observed during exhalation, respiratory rate divided by tidal volume, duration in seconds required to inspire 67% of tidal volume of inspiration, and tidal volume of inspiration divided by maximum flow value observed during inspiration.

4. The pressure support system of claim 1, wherein the number of breath features include at least one of: minimum patient flow during expiration divided by a pressure difference during expiration, maximum patient flow during inspiration divided by a pressure difference during inspiration, tidal volume of inspiration divided by the pressure difference during inspiration, and tidal volume of expiration divided by the pressure difference during expiration

5. The pressure support system of claim 1, wherein the comfort level is calculated based on the following equation:
Y=M1X1+M2X2+M3X3 . . . +B
where Y is the comfort level, X1, X2, X3 are each values of one of the number of breath features, M1, M2, M3 are coefficients corresponding to the number of breath features, and B is a bias value.

6. The pressure support system of claim 5, wherein the coefficients corresponding to the number of breath features and the bias value are based on analysis of experimental data.

7. The pressure support system of claim 1, wherein the processing unit is structured to compare the calculated comfort level to a target comfort level and to raise the gain of the airflow generator if the calculated comfort level is below the target comfort level and to lower the gain of the airflow generator if the calculated comfort level is above the target comfort level.

8. A method of providing pressure support therapy to a patient, the method comprising:
generating a flow of breathing gas to the patient;
sensing characteristics of breaths of the patient;
calculating a number of breath features of the patient based on the characteristics of breaths of the patient;
calculating a comfort level based on one or more of the calculated number of breath features; and
adjusting a gain of the flow of breathing gas to the patient based on the calculated comfort level.

9. The method of claim 1, wherein the comfort level of the patient is a representation of the patient's perceived comfort with a level of pressure compensation provided in the pressure support therapy.

10. The method of claim 8, wherein the number of breath features include at least one of: asymmetry of a patient inspiration flow waveform, tidal volume of inspiration divided by time of inspiration, inspiration time divided by exhalation time, exhaled tidal volume divided by a minimum patient flow observed during exhalation, respiratory rate divided by tidal volume, duration in seconds required to inspire 67% of tidal volume of inspiration, and tidal volume of inspiration divided by maximum flow value observed during inspiration.

11. The method of claim 8, wherein the number of breath features include at least one of: minimum patient flow during expiration divided by a pressure difference during expiration, maximum patient flow during inspiration divided by a pressure difference during inspiration, tidal volume of inspiration divided by the pressure difference during inspiration, and tidal volume of expiration divided by the pressure difference during expiration

12. The method of claim 8, wherein the comfort level is calculated based on the following equation:
Y=M1X1+M2X2+M3X3 . . . +B
where Y is the comfort level, X1, X2, X3 are each values of one of the number of breath features, M1, M2, M3 are coefficients corresponding to the number of breath features, and B is a bias value.

13. The method of claim 12, wherein the coefficients corresponding to the number of breath features and the bias value are based on analysis of experimental data.

14. The method of claim 8, further comprising:
comparing the calculated comfort level to a target comfort level; and
raising the gain of the airflow generator if the calculated comfort level is below the target comfort level and lowering the gain of the airflow generator if the calculated comfort level is above the target comfort level.

15. A non-transitory computer readable medium storing one or more programs, including instructions, which when executed by a computer, causes the computer to perform a method of providing pressure support therapy to a patient, the method comprising:
generating a flow of breathing gas to the patient;
sensing characteristics of breaths of the patient;
calculating a number of breath features of the patient based on the characteristics of breaths of the patient;
calculating a comfort level based on one or more of the calculated number of breath features; and
adjusting a gain of the flow of breathing gas to the patient based on the calculated comfort level.
